(19) United States
(12) Patent Application Publication
(10) Pub. No.: US 2016/0175065 A1
(43) Pub. Date: Jun. 23, 2016

(54) MULTIFUNCTIONAL TELESCOPING CLEANING DEVICE

(71) Applicant: COVIDIEN LP, Mansfield, MA (US)

(72) Inventors: Ricardo Alexander Gomez, Lighthouse Point, FL (US); Sandy Lawrence Heck, Los Angeles, CA (US); Eric William Conley, South Berwick, ME (US); Trisian Ian Jordan, Ft. Lauderdale, FL (US)

(21) Appl. No.: 14/906,745

(22) PCT Filed: Jul. 24, 2014
(86) PCT No.: PCT/US2014/048017

§ 371 Date: Jan. 21, 2016

Related U.S. Application Data
(60) Provisional application No. 61/858,168, filed on Jul. 25, 2013.

(51) Int. Cl. A61B 90/70; B08B 9/035

(52) U.S. Cl. CPC A61B 90/70; B08B 9/035

(57) ABSTRACT

Multifunctional telescoping cleaning devices and methods of using the devices are provided for cleaning the interior of surgical trocars and cannulas. The multifunctional cleaning device may include a telescoping feature which may be used in bariatric procedures where reach is important. The device may also be configured in some instances to clean all standard trocars and cannulas sized between 5-12 mm with depth of up to 50 cm. The device may have porous cleaning absorbent members. The device may also include the ability to perform aspirating, dissecting, filtering, irrigating and evacuating in surgical laparoscopic procedures through a fluid flow passage in the telescoping tool. The device may quickly and efficiently cleans trocars, cannulas, and laparoscopic lens.

### CROSS-REFERENCE TO RELATED APPLICATIONS

This application is entitled to the benefit of and incorporates by reference subject matter disclosed in the International Patent Application No. PCT/US2014/048017 filed on Jul. 24, 2014 and U.S. Provisional Patent Application No. 61/858,168 filed on Jul. 25, 2013.

### TECHNICAL FIELD

The present invention relates to a laparoscopic multifunctional telescoping cleaning device and a method for use in cleaning, dissecting, irrigation, filtration, traction, and evacuation in surgical laparoscopic procedures.

### BACKGROUND

Laparoscopic surgery sometimes called Keyhole or “Minimally Invasive Surgery” (MIS) is a relatively new type of surgery recently introduced into the general surgical field in 1988. It involves the introduction of small incisions in a patient (usually between 0.5 and 1.5 cm) whereby access to the thoracic, abdominal or pelvic cavities is obtained by the use of a medical device called a trocar. The word trocar comes from the French trois-carre meaning three-edged or triangular point. Trocars typically have a three edged point at their distal end which is used for inserting through the skin layer and into the cavity area of a patient. A trocar is often used in combination with another surgical tool called a cannula.

A cannula is a hollow cylinder or tube that is inserted into the body to deliver or remove fluids. Prior to starting a surgical procedure, a small incision is performed on the patient whereby a trocar is inserted. Care must be taken in choosing the location of entry to prevent accidental severing of blood vessels such as the veins and arteries of the anterolateral abdominal wall. A procedure called trans illuminating is sometimes used to prevent this problem. This is a procedure whereby light is shined from inside the abdominal wall clearly illuminating where the blood vessels are located. Illumination allows the surgeon to determine the best approach for performing the medical procedure, thus, minimizing blood loss and trauma to the patient. An insufflator also referred to as a pump, is then used to inflate the cavity area with carbon dioxide (CO2), thus, providing a means of creating greater space by expanding the abdominal walls. This expansion provides better viewing for the surgeon during the medical procedure. A special medical device called a laparoscope is subsequently inserted through the trocar/cannula allowing the surgeon to look inside the cavity area.

A laparoscope is the main medical tool used in Laparoscopic or Minimally Invasive Surgery (MIS). During this procedure, there is a constant insertion and removal of surgical tools through the trocar/cannula device. These exchanges comprise between 10 to 30 percent of the total procedure time. Minimizing these exchanges has many benefits to the patient among them are: the reduction of surgical time, operating costs, trauma and amount of anesthesia required to keep the patient sedated. The surgeon also benefits by reducing the overall operating time. The introduction of multifunctional devices is a popular way to reduce the number and time of surgical tool exchanges.

Trocars often have small ports that range in size from 5-12 mm used in the insertion of medical devices. The deciding factor in choosing the proper trocar size varies, but is primarily decided by the size of the instruments, the procedure used, and the surgeon's individual preferences. The standard trocar length used today is 10 cm, with extra-long trocars of 15 cm used in bariatric surgery. Bariatric surgery is often performed on obese people who have thick or extra layers of fat, requiring longer surgical tools to access, deliver, and clean the affected area. Larger sized ports of entry are preferred because small ports impede the timely exchange of surgical tools and inhibit the surgeon's dexterity. These larger ports provide access to larger surgical tools needed for dissecting and retrieving the larger tissue specimens. These larger specimens would typically not pass through narrower trocar/cannulas.

Surgical tools include, but are not limited to: trocars, cannulas, evacuating devices, irrigation devices, laparoscopes, dissectors, grabbers, forceps, cautery's, probes and ligating appliers. In this constant interaction of surgical tools being inserted and removed through the trocar, deposits of blood, tissue and other bodily fluids can adhere to the trocar/cannula or laparoscope. These accumulated residues often blur or completely block the viewing area of the surgeon requiting the temporary stopping of the surgical procedure giving greater rise to peril. The procedure continues once the medical device has been cleaned or unblocked.

For example in laparoscopic urology surgery, it is common practice to use multiple tools such as aspiration, irrigation, cauterization, and dissection tools. The goal is to complete the surgery as quickly as possible minimizing blood loses. The problem with this approach is that the constant interchanging of tools increases the overall surgical time. In order to help reduce the surgical time, multifunctional tools are used. Hemostasis is the process which causes bleeding to stop. Some surgical devices deliver temporary hemostatic agents to a particular area thus reducing blood loss. The drawback to this is that the irrigation process tends to encompass a broader area than intended. This causes unintended side effects to the surrounding areas. There is a need in the field for multifunctional tools and a method of specific delivery of hemostatic agents.

There exists in the field a need for an improved cleaning device.

### SUMMARY

The present device overcomes the deficiencies of the known art and the problems that remain unsolved by providing a method and apparatus for a telescoping multifunctional cleaning device.

The telescoping multifunctional cleaning device includes a plurality of telescoping tubes with an absorbent member disposed on each end of the device. The device also includes a locking mechanism for locking the telescoping tubes in place. The telescoping tubes allow overall length adjustment of the device such that the device can be used in various types of surgeries and situations. The absorbent members can be of varying sizes to accommodate cleaning of medical devices of varying sizes. The device can be connected to a suction source or a fluid source to facilitate passage of fluids therethrough.

The foregoing summary is illustrative in nature and is not intended to be in any way limiting. In addition these and other aspects, features, and advantages of the present device will become more readily apparent from the attached drawings and the detailed description of the embodiments.

### BRIEF DESCRIPTION OF THE DRAWINGS

In order to better explain the characteristics of the present device, the embodiments will hereinafter be described in conjunction with the drawings provided to illustrate and not to limit the device, in which:

FIG. 1 is a sectional view of an exemplary laparoscopic multifunctional telescoping cleaning device;

FIG. 2 is an exploded assembly view of the exemplary laparoscopic multifunctional telescoping cleaning device;

FIG. 3 is an exploded view of an alternative embodiment of the laparoscopic multifunctional telescoping cleaning device;

FIG. 4 is a sectional view of proximal and distal ends of an alternative embodiment of the laparoscopic multifunctional telescoping cleaning device;

FIG. 5 is an isometric sectional view of an embodiment locking mechanism;

FIG. 6 presents a sectional view of an alternative embodiments locking mechanism; and

FIGS. 7A/7B are perspective views of an alternative embodiment of an adhesive locking mechanism.

The drawings are included to provide a further understanding of the disclosure, and are incorporated into and constitute a part of this specification. The drawings illustrate various embodiments of the disclosure and together with the description serve to explain the principles and operations of the disclosure. Like reference numerals refer to like parts throughout the several views of the drawings.

### DETAILED DESCRIPTION

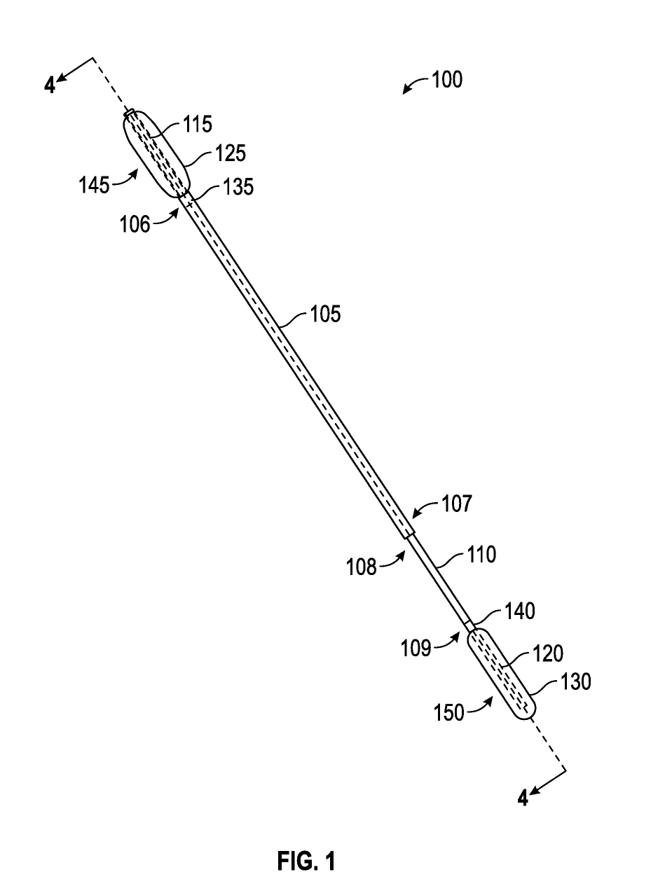

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, ‘.vertical”, “horizontal”, and derivatives thereof shall relate to the device as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence. Specific dimensions and other physical characteristics relating to the embodiments disclosed herein are not to be considered as limiting, unless the claims expressly state otherwise.

A multifunctional telescoping laparoscopic cleaning device, or wipe, of FIG. 1 extends from a proximal end 145 to a distal end 150 and includes two or more hollow telescoping tubes 105,110 with the telescoping tubes 105, 110 fitting within each other. Each of the telescoping tubes 105, 110 includes a proximal end 106, 108 and a distal end 107, 109, respectively.

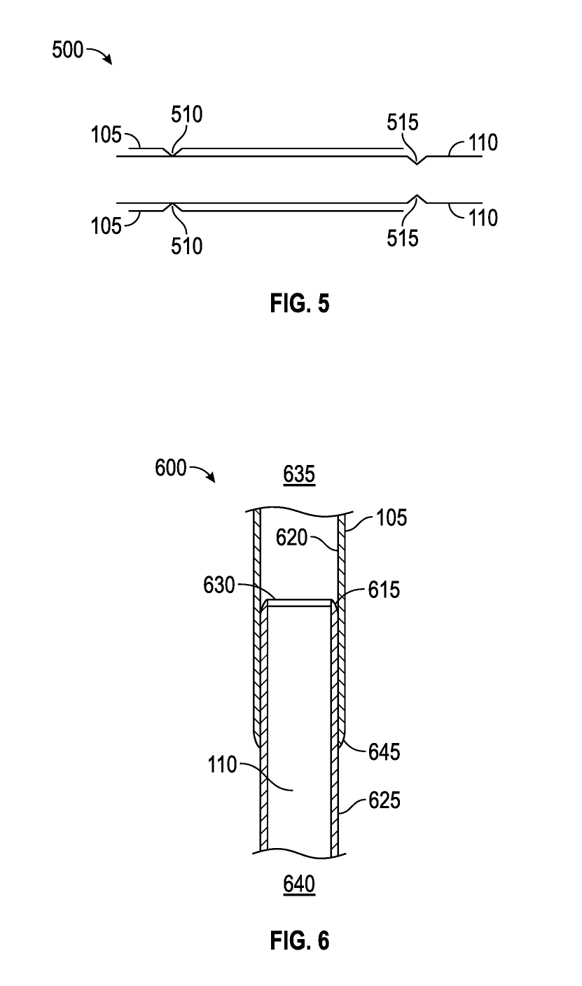

One telescoping tube is slid relative to the other telescoping tube altering the device's overall length. The telescoping tubes 105,110 have different diameters. The outer diameter of the distal telescoping tube 110 is slightly smaller than the inner diameter of the larger proximal telescoping tube 105. Once the length of the multifunctional laparoscopic device 100 has been chosen, the device maintains its length by means of a locking mechanism 500, 600, 700 as shown in FIG. 5, 6, or 7B. Numerous different possibilities for the locking mechanism exist.

With reference to FIG. 1, two attachment tubes 115, 120 may be threaded or glued to their respective telescoping tubes 105,110. The proximal and distal telescoping tubes are the primary determinants for choosing the size of absorbent members 125, 130. When threaded screws 135, 140 of the telescoping tubes 105, 110 are used, they allow the user to change the size of the absorbent members 125, 130 to the particular trocar/cannula opening desired. The absorbent members 125, 130 can be fastened to the telescoping tubes 105, 110 through gluing, adhesives, heat shrinking or any number of well established procedures well known in the field. Alternatively, the absorbent members 125, 130 can be attached directly onto the telescoping tubes 105, 110.

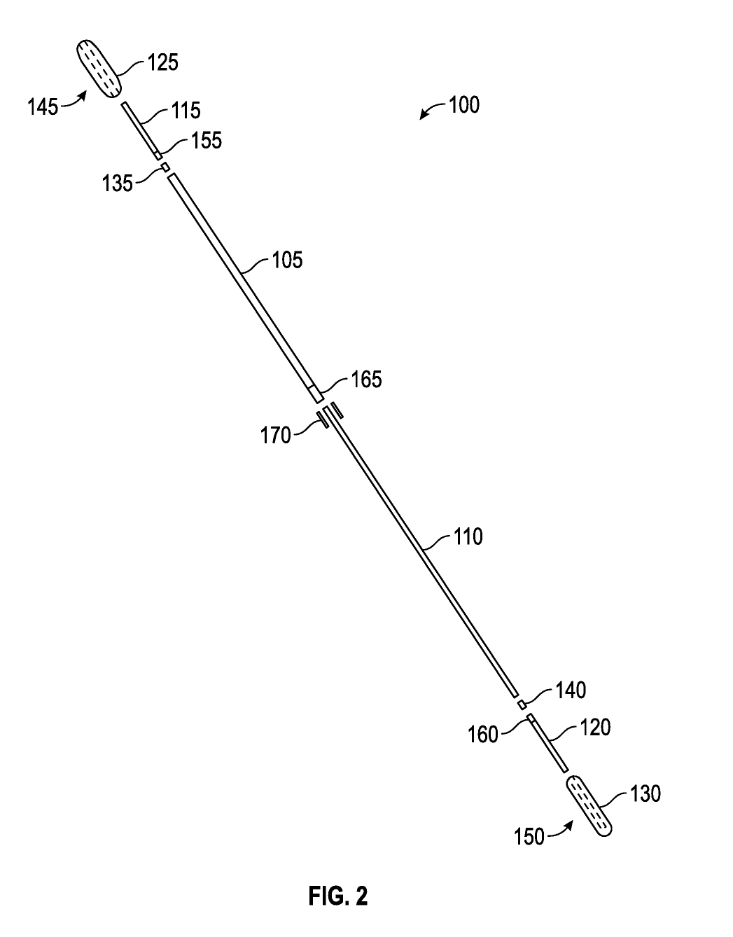

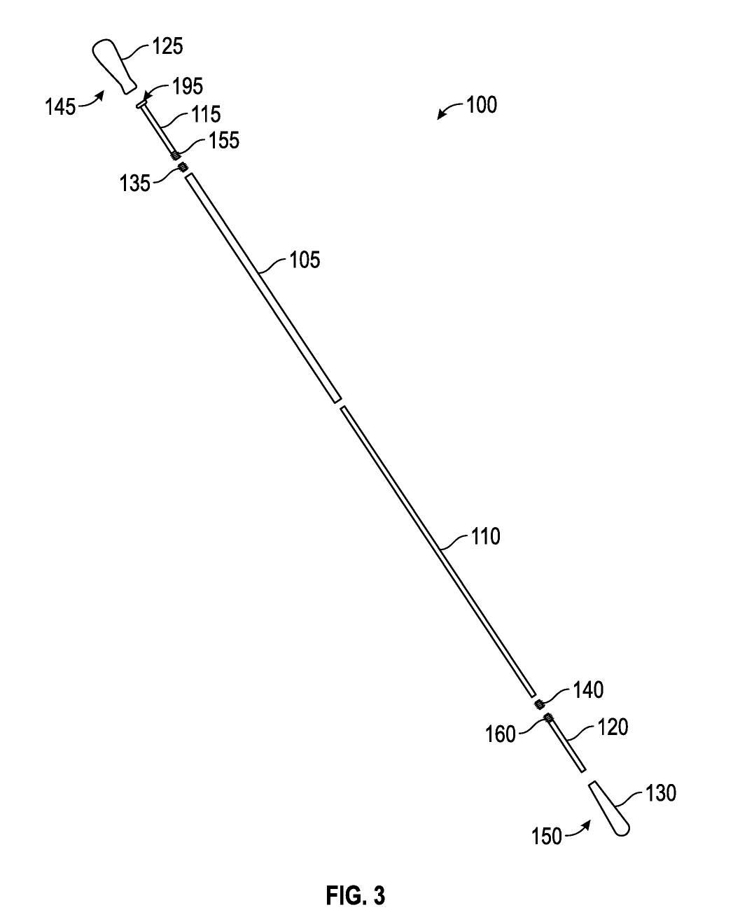

With reference to FIG. 2, proximal and distal attachment tubes 115, 120 are securely attached into the telescoping tubes 105, 110 to make the final device. Methods of connecting the attachment tubes 115,120 to the telescoping tubes 105,110 include but are not limited to: heating the attachment tubes 115,125 past their melting temperature and then pressing the attachment tubes 115,120 onto the telescoping tubes 105,110. Other methods used could be, mechanically joining the tubes with fasteners, intermediary materials such as heat shrink materials, or integrally forming the telescoping tubes 105,110 with the attachment tubes 115,120. Still another method could be the use of plastics, metals or composite materials interconnected with the use of a threaded screw or end 155, 160 and screw insert 135, 140 such as also seen in FIG. 3, whereby the telescoping tubes 105,110 are connected to the attachment tubes 115, 120. Methods that are generally practiced and well known in the art can also be used to join the two pieces of members.

Each telescopic tube 105, 110 can have a hole provided near the proximal 145 or its distal end 150 for joining the attachment tubes 115, 120 to the telescopic tubes 105, 110. The attachment tubes 115, 120 can have protrusions 510 similar to those shown in FIG. 5, on its inner surface that fit into a corresponding recess of the telescopic tubes 105, 110. Thus, mating locking features may be formed in the telescoping tubes 105, 110 and attachment tubes 115, 120 to facilitate attachment. A crimped end 165 may be used with the proximal telescoping tube 105, forcing the underlining brass members 170 to lock it in place when fully extended.

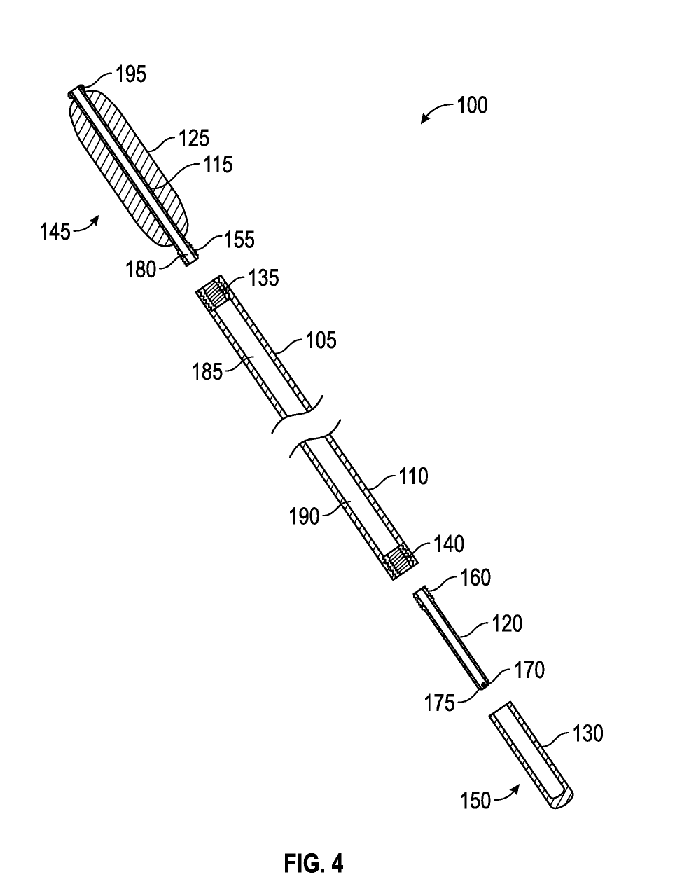

With reference to FIG. 3, an alternative embodiment of the telescoping multifunctional laparoscopic wipe 100 includes a proximal attachment tube 115 used for a larger sized trocar/cannula. The proximal attachment tube 115 includes a bulbous rounded portion 195 and a threaded end 155. The attachment tube can be made from any material not limited to but including plastics, stainless steel, metals, or composite materials. In some instances the attachment tube may be made from polyethylene such as HDPE. The bulbous rounded portion 195 is shaped for easy attachment to a suction source (not shown). When attached to the suction source, the suction source tubing (also not shown) is pressed over the bulbous rounded portion 195 until the tubing tightly engages. The tubing is tightly sealed around the proximal attachment tube 115 and will prevent any blood or fluid from leaking from the multifunctional laparoscopic wipe 100. The suction source can be connected to the multifunctional laparoscopic wipe 100 with tubing capable of permitting the flow of blood and other fluids from the body through hollow passageways 180, 185, 190, as shown in FIG. 4, out to a holding vessel. Proximal attachment tube 115 is hollow and receives a proximal absorbent member 125 on its outer surface. The absorbent member 125 is of sufficient size to clean the largest trocars commonly encountered in laparoscopic surgery today. The proximal absorbent member 125 may be sized so as to clean the diameter of a 12 mm trocar/cannula and laparoscopic lenses. The proximal absorbent member 125 can also be used to abrade tissue materials. The proximal absorbent members 125, 130 can have a variety of shapes that enhance their surface area. In an alternate embodiment, insert screws 135, 140 attach to proximal and distal attachment tubes 115, 120, allowing different sized absorbent members to be selected.

With reference to FIG. 4, in an alternative embodiment of the multifunctional laparoscopic wipe 100, the distal attachment tube 120 is used for cleaning smaller sized trocars. The proximal and distal attachment tubes 115, 120 are substantially cylindrical with threaded ends 155,160. Screw inserts 135, 140 are used to hold it in place. In one embodiment, no screws are used and other methods of attachment can be used. The distal cylindrical portion's outer surface receives the smaller distal sized absorbent member of the multifunctional laparoscopic wipe. The hollow absorbent member 130 is slid over the distal telescopic tube 120 and then formed onto the multifunctional laparoscopic wipe. The positioning of attachment tube holes 170 is important because they allow for fluid to enter the hollow canal of the telescopic tubes and allow fluid to flow towards the exit. The distal end of the attachment tube 120 has features for suctioning liquids such as blood. These features include circular holes 170 located circumferentially on the outer surface of the distal attachment tube 120. The circular holes 170 provide a fluid path for blood or other bodily fluids to enter the hollow cylindrical tube portion of the distal attachment tube 120. The holes 170 increase the effective suctioning of liquids from the affected area. The distal end of the attachment tube has a rounded or chamfered edge 175. The smooth shape of the edge reduces pressure created when the multifunctional laparoscopic wipe 100 is manipulating tissue or organs. The rounded edge also reduces the potential tearing of the absorbent member 130.

Proximal absorbent member 125 need not extend past the bulbous rounded portion 195 of proximal attachment tube 115. Absorbent member 125 may be the same length as its attachment tube 115 in some instances. The absorbent members 125,130 fit into different sized trocar/cannulas (not shown) and make contact with and/or rub against the interior surface of the trocar/cannula for providing the best cleaning effect. The material for absorbent member 125,130 should be porous enough that fluid can flow through the surface and into the telescopic tubes 105,110 when the multifunctional laparoscopic device 100 is performing suction procedures. Depending on the materials used for the attachment member, the absorbent member can be attached, but is not limited to, being glued, pressed together with the attachment tube, or integrally formed onto the attachment tube, etc. The diameters of the absorbent members 125,130 may be selected to allow the cleaning of trocar/cannula diameters between 5-12 mm.

With reference to FIG. 5, a locking mechanism 500 for the proximal and distal telescoping tubes 105,110 includes features, or deformations, 510, 515 formed in walls of the telescoping tubes 105, 110. Each feature, or as shown in FIG. 5, a deformation, 510, 515 has a depression which forms a valley in the tube wall. The deformations can either encircle the complete diameter of the telescoping tubes or be formed intermittedly. The inner telescoping tube 110 has a slight taper which allows the outer tube 105 to slide within one another without interference. The protrusion 510 of the outer tube 105 and the inner tube recess wall 515 combine to lock the device in place. This is only one of many ways of attaching two medal tubes. The number of deformations used when manufactured can vary. The distance between the deformations can also be varied. Different numbers of telescoping tubes may also be used with locking mechanisms. If more than one locking point of the multifunctional laparoscopic wipe 100 is desired, one of the telescoping tubes can include multiple deformations to allow adjustability and selection of different overall lengths of the device. The other telescoping tube may need one protrusion because this protrusion can correspond with multiple deformations of the inner telescoping tube, corresponding with different lengths. Multiple deformations of the telescoping tube enhance the adjustability of the multifunctional laparoscopic wipe 100. The last deformation of each tube should be located close to the end of the telescopic tubes 105,110 to achieve the maximum length of the multifunctional laparoscopic wipe 100. When force is applied, protrusion 510 should correspond with recessed areas 515 preventing the movement of the telescopic tubes 105, 110. The locking force may be strong enough to resist potential collapsing of the multifunctional laparoscopic wipe 100 during a surgical procedure, but weak enough to allow adjustability. In this embodiment, the shape of the deformation is a V shape, but the deformations can be varied to adjust the locking force of the telescoping tubes.

With reference to FIG. 6, a bushing locking mechanism 600 of the multifunctional laparoscopic device 100 includes a bushing 615. The bushing 615 is attached onto an outer surface 625 near distal end 640 of inner telescopic tube 110 and makes contact with outer telescopic tube 105. The bushing 615 makes contact with the outer telescopic tube 105 along its inner wall surface 620. The bushing 615 is shaped to conform to the outer telescopic tube 105 having a beveled outer telescoping tube end 645. The movement of the inner telescopic tube 110 having a beveled tip 630 is restricted by the frictional force created by the bushing 615 as it contacts the inner wall surface 620 of the outer telescopic tube 105, The frictional forces prevent movement of the telescopic tubes 105, 110 unless the surgeon or surgical assistant applies a larger force than the friction. The frictional force has to be strong enough to hold the telescoping multifunctional laparoscopic wipe 100 in place, during the abrasion process of tissue dissection within the body, the cleaning process of the laparoscope lens, or the insertion of the multifunctional laparoscopic wipe 100 through a trocar/cannula device.

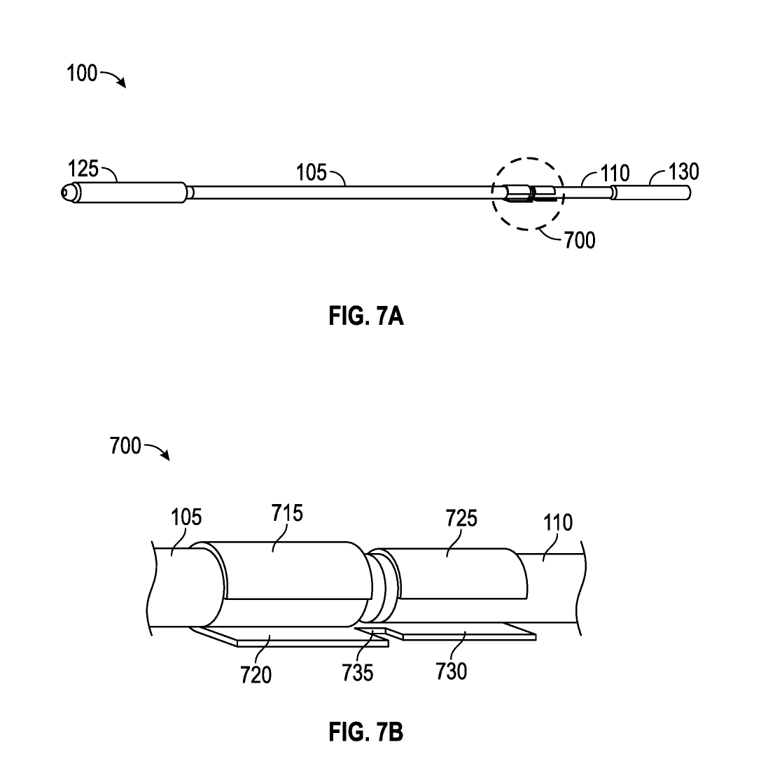

With reference to FIGS. 7A-7B, another embodiment of the multifunctional laparoscopic wipe 100 includes an attachable adhesive strip 700. The adhesive strip 700 acts as a locking mechanism to prevent the telescoping tubes 105, 110 from moving relative to one another during a surgery. The adhesive strip 700 has proximal and distal upper adhesive portions 715,725. The proximal adhesive portion 715 is pre-attached to a proximal telescopic tube 105 outer surface. The distal upper adhesive portion 725 is not pre-attached to the telescopic tube 110. This is done so that the adhesive tape 700 can slide over the distal telescoping tube 110 prior to selecting a proper length. There is space between upper curved portions 725 inner surface and the distal telescopic tube 110 to allow the outer proximal telescopic tube 110 to slide freely to the desired length prior to curved position 715 attaching to proximal telescopic tube 105. Flat portions 720, 730 have an adhesive flat surface backing. Bottom curved portion 715 also has an adhesive covered inner surface with backing. Once the multifunctional laparoscopic wipe 100 is of the desired length, all backing is removed and the adhesive surfaces are pressed into contact with the telescopic tubes 105, 110 outer surface. Bridge 735 helps prevent the telescopic tubes from separating, by locking the multifunctional laparoscopic wipe at the desired length. Adhesive strip bridge 700 is connected to both of the flat surfaces of the adhesive strip 720,730.

The device can be opened at the beginning of the surgical procedure and kept ready for surgical staff on a sterile back table or may be opened during the surgical procedure as needed. The device is operated by holding one end through a trocar. The device may be pushed in and pulled out of the trocar as needed to clean fluids, and other debris that have collected along the circular internal wall of the trocar. In some instances the device may be designed for a single patient use and disposed of at the end of each procedure, but the device may be used repeatedly over the course of a single procedure until it becomes saturated with fluids other debris. The device may have an X-ray detectable element embedded in the sponges, connecting rod or both.

The material of the sponges can be foam, fabric or any other material or combination of materials that are both durable and absorptive, such as a microfiber material. This can be a piece of microfiber fabric folded, pressed or rolled onto itself, or it could be a surface of microfiber cloth over a foam, cotton or rayon core. What constitutes the microfiber material is the D.P.F. or denier per filament. The microfiber material may have a D.P.F. from about 0.05 to about 1.5. The absorptive sponge can take on numerous shapes.

Proximal attachment tube 115 and distal attachment tube 120 can be screwed into or permanently attached to the telescoping tubes 105, 110 thus enabling the foam/fabric material heads to be replaced to compensate for specific trocar/cannula sizes.

The laparoscopic multifunctional telescoping cleaning device can also be of a unitarian construction with absorbent members permanently attached at the ends used in the cleaning of trocar/cannula devices.

The multifunctional laparoscopic wipe also referred to as the wipe is a disposable adjustable device that cleans multiple laparoscopic devices. It may clean trocar cannulas of diameters between 5-12 mm in some instances. The device may have an extended reach of between 15-50 cm in some instances. The extended length in some instances may be selected to be between 20-24 cm, though other ranges may be used in different instances. The device cleans different types and sizes of trocars including bariatric trocars.

Both ends of the multifunctional laparoscopic wipe have absorbent members for cleaning the inside of a trocar cannula or a laparoscope lens. The two absorbent members have different diameters, but are of similar lengths. The smaller diameter absorbent member can be sized to clean the inner cannula of the smallest trocar in laparoscopic surgery. In general, the absorbent member is sized to clean a 5 mm trocar. The smaller diameter absorbent member is inserted through the abdomen wall when it is necessary to suction or remove fluids from within the body. The larger proximal absorbent member can clean various trocar cannula inner diameter sizes prevalently used in laparoscopic procedures. In some instances, the absorbent member is of sufficient diameter to clean a 12 mm trocar. The absorbent members can be made from but are not limited to absorbent materials such as polyurethane foam, microfiber, or cotton. Both absorbent members can have a reflective or shiny surface for enhanced visibility. The color of the absorbent members can be chosen for high contrast with bodily tissue. In some instances, white foam may be used to make it easier for the surgeon to see any attached materials. The absorbent members fit easily through the trocar cannula of the corresponding size. The absorbent members have sufficient surface area for repeatedly cleaning the laparoscope lens during a laparoscopic procedure.

Furthermore, the multifunctional laparoscopic wipe performs suctioning of blood and fluids through the telescopic tubing. One end of the multifunctional laparoscopic wipe is shaped to attach to a suction source. The other end of the multifunctional laparoscopic wipe has holes or fenestrations for the passage of blood and other fluids into the hollow telescopic tubing. The suction strength of the multifunctional laparoscopic wipe is varied by covering an opening located on the outside of the telescopic tubing. The opening may be located on the section of the telescopic tubing that contains the suction attachment. The opening is similar in size to a surgeon's thumb or finger. The surgeon alters the suction strength by placing his thumb or finger over the opening.

Additionally, the multifunctional laparoscopic wipe can serve as an irrigation/evacuation tool. Since the shaft of the multifunctional laparoscopic wipe is hollow, fluid can travel through the interior shaft of the device that also provides a secure sealed passage for fluids to flow. When fitted with a mechanism for attaching the hollow shaft to a source of fluid, the multifunctional laparoscopic wipe can act as a laparoscopic irrigator. The multifunctional laparoscopic wipe can be connected to a source of irrigation fluid such as a sterile water, or saline solution by means of tubing.

Further, the irrigation function can supply a heated fluid for enhancing the cleaning effect and/or defogging of the multifunctional laparoscopic wipe on a laparoscope lens. The heated fluid can contain a surfactant for breaking up fats and oils left on the laparoscope lens surface.

Additionally, the multifunctional laparoscopic wipe changes its length by the relative motion of the multiple telescoping tubes. The telescoping tubes are rigid and made of hollow thin wall tubing. The tubes may be made from stainless steel in some instances, but other materials including plastics or composite materials are possible. The telescoping tubes are x-ray detectable within the human body. Once the multifunctional laparoscopic wipe is expanded to the surgeon's desired length, the surgeon locks the telescoping tubes with the locking mechanism. In the locked position, the telescoping tubes will not collapse when the multifunctional laparoscopic wipe is deployed within the body. The locking of the telescoping tubes prevents the absorbent member or cleaning head from moving when it contacts another object such as a laparoscope lens or body tissue (when abrading the tissue).

Formed over each telescopic tube is another tube. The two outer tubes of the multifunctional laparoscopic wipe include the attachment surfaces for the absorbent members and have specialized functions. The outer tube corresponding to the distal smaller cleaning end of the multifunctional laparoscopic wipe has holes or fenestrations around its perimeter. The evacuating holes or fenestrations allow blood or fluid to enter into the multifunctional laparoscopic wipe. The proximal end has a separate outer tube formed over the inner telescoping tube. The outer tube formed on this end of the multifunctional laparoscopic wipe is designed for attaching up to a 12 mm foam member and a suction tube. Unlike the distal 5 mm tube, the proximal tube does not contain fenestrations or holes. The outer tube is hollow with a solid outer wall. One end of the outer tube has a protrusion for attaching a piece of tubing to the multifunctional laparoscopic wipe. The protrusion has a large enough diameter that tubing can be attached to the device, but a small enough diameter so that the device can fit into a 12 mm diameter trocar.

The device also has the ability to apply anti-fogging liquid used to clean medical scopes and other medical items before, during and after medical procedures. An x-ray detectable element may be coupled to at least one of the connecting rods, absorbent sponge, or second absorbent sponge.

A surgical device may include two or more telescoping tubes having a fluid flow passage. The fluid flow passage may extend through a length of the surgical device. A first of the tubes may have a fluid inlet. A second tube may have a fluid outlet. Two or more absorbent members may be attached to the telescoping tubes. At least one of the absorbent members may at least partially cover at least one of the fluid inlet and the fluid outlet. Each absorbent member may be sized to rub against an inner surface of a different sized surgical port cannula when the surgical device is inserted therein.

The surgical device may include a locking mechanism to prevent collapsing of the telescoping tubes. The locking mechanism may include a first feature formed in the first telescoping tube matingly engaging with a second feature formed in the second telescoping tube as discussed herein. In some instance two or more of the first and/or second features may be formed in different positions in order to prevent collapsing of the telescoping tubes in each of the different positions while still allowing an adjustability of an overall length of the device.

In some instances, the locking mechanism may include a bushing on the first telescoping tube and a fitting on the second telescoping tube engaging with the bushing. In some instances, the locking mechanism may include an adhesive strip securing the first telescoping tube to the second telescoping tube.

In some instances, at least one intermediate telescoping tube may be disposed between the first telescoping tube and the second telescoping tube.

In some instances, the first telescoping tube may include a threaded attachment tube component containing at least one of the absorbent members. The threaded attachment tube component may be selectively detachable from the first telescoping tube and replaceable with another threaded attachment tube component containing a different sized absorbent member.

One or more of the absorbent members may have a porosity that is large enough to allow one or more fluids to pass through. One or more of the absorbent members may attach to the telescoping tubes via attachment members.

The surgical device may include an irrigation mode and/or a suction mode. In an irrigation mode, the fluid inlet may be connected to a fluid source. In a suction mode, the fluid outlet may be connected to a suction source.

In the irrigation mode, at least one of the absorbent members may cover the fluid outlet. Fluid may be sent from the fluid source through the fluid flow passage to the at least one absorbent member covering the fluid outlet.

In the suction mode, at least one of the absorbent members may cover the fluid inlet. Fluid may be sucked through the at least one absorbent member covering the fluid inlet into the fluid flow passage to the fluid outlet.

A bulbous portion may be disposed on at least one of the fluid inlet and the fluid outlet. The bulbous portion may be designed and/or adapted for respective attachment to the fluid source or the suction source.

In some instances, at least one end of at least one of the telescoping tubes may include a chamfered edge. In some instances the second telescoping tube may slidably fit into the first telescoping tube.

In some instances, the surgical device may be a minimally invasive surgical device having a device proximal end and a device distal end. This device may include a first telescoping tube having a first tube proximal end and a first tube distal end. The first telescoping tube may be disposed on the device proximal end. The second telescoping tube may have a second tube proximal end and a second tube distal end. The second telescoping tube may be disposed on the device distal end. The second tube proximal end may slidably fit into the first tube distal end. A first absorbent member may be disposed on the proximal end of the first telescoping tube. A second absorbent member may be disposed on the distal end of the second telescoping tube. The first telescoping tube and the second telescoping tube may be adjusted with respect to each other to vary the overall length of the device and lock with respect to each other once the overall length has been determined. This surgical device may include at least one hole to allow passage of fluids through the device when the device is attached to a suction source or a fluid source. In some instances, the absorbent members may be of different sizes between about 5 and 12 millimeters to allow cleaning of cannulas of those sizes during minimally invasive surgery.

A method may be performed for cleaning a cannula in a minimally invasive surgical port. Telescoping tubes having a fluid flow passage extending therethrough may be adjusted to a predetermined telescopic length. An end of the telescoping tubes may be inserted into a surgical port cannula. The inserted end may have an absorbent member associated with it that may be sized to rub against an inner surface of the surgical port cannula when the surgical device is inserted therein. A noninserted end of the telescoping tubes may have a different sized absorbent member associated with it. A fluid flow through the fluid flow passage may be triggered or otherwise caused to occur at least while the end of the telescoping tubes is inserted in the surgical port cannula.

Flowing fluid may pass through the absorbent member associated with the inserted end of the telescoping tubes while the end is inserted in the surgical port cannula. A suctioning of fluid in the surgical port cannula through the absorbent member and the fluid flow passage may be activated while the inner surface of the surgical port cannula is rubbed clean with the same absorbent member associated with the inserted end of the telescoping tubes as the end is inserted in the surgical port cannula.

Irrigation of the fluid flow passage may be initiated with fluid from an external source while the end of the telescoping tubes is inserted in the surgical port cannula. Irrigation of the absorbent member associated with the inserted end of the telescoping tubes may also be initiated with the external source fluid in the fluid flow passage while the end is inserted in the surgical port cannula. The external source fluid irrigated on the absorbent member may be applied to an inner surface of the surgical port cannula while the inner surface of the surgical port cannula is also rubbed clean with the same absorbent member.

The above-described embodiments are merely exemplary illustrations of implementations set forth for a clear understanding of the principles of the invention. Many variations, combinations, modifications or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

1. A surgical device comprising:
a plurality of telescoping tubes having a fluid flow passage through the surgical device, a first of the tubes having a fluid inlet and a second having a fluid outlet; and
a plurality of absorbent members attached to the telescoping tubes, at least one of the absorbent members at least partially covering at least one of the fluid inlet and the fluid outlet, each absorbent member sized to rub against an inner surface of a different sized surgical port cannula when the surgical device is inserted therein.

2. The device according to claim 1, further comprising a locking mechanism to resist collapsing of the telescoping tubes.

3. The device according to claim 2, wherein the locking mechanism comprises a first feature formed in the first telescoping tube matingly engaging with a second feature formed in the second telescoping tube.

4. The device according to claim 3, further comprising a plurality of first or second features formed in different positions that resist collapsing of the telescoping tubes in each of the different positions while allowing adjustability of an overall length of the device.

5. The device according to claim 2, wherein the locking mechanism comprises a bushing on the first telescoping tube and fitting on the second telescoping tube engaging with the bushing.

6. The device according to claim 2, wherein the locking mechanism comprises an adhesive strip securing the first telescoping tube to the second telescoping tube.

7. The device according to claim 1, further comprising at least one intermediate telescoping tube disposed between the first telescoping tube and the second telescoping tube.

8. The device according to claim 1, wherein the first telescoping tube includes a threaded attachment tube component containing at least one of the absorbent members and the threaded attachment tube component is selectively detachable from the first telescoping tube and replaceable with another threaded attachment tube component containing a different sized absorbent member.

9. The device according to claim 1, wherein the absorbent members have a porosity allowing fluid to pass through.

10. The device according to claim 1, wherein the absorbent members are attached to the telescoping tubes via attachment members.

11. The device according to claim 1, wherein in an irrigation mode the fluid inlet is connected to a fluid source and in a suction mode the fluid outlet is connected to a suction source.

12. The device according to claim 11, wherein in the irrigation mode at least one of the absorbent members covers the fluid outlet and fluid is sent from the fluid source through the fluid flow passage to the at least one absorbent member covering the fluid outlet.

13. The device according to claim 11, wherein in the suction mode at least one of the absorbent members covers the fluid inlet and fluid is sucked through the at least one absorbent member covering the fluid inlet into the fluid flow passage to the fluid outlet.

14. The device according to claim 11, further comprising a bulbous portion disposed on at least one of the fluid inlet and the fluid outlet, and adapted for respective attachment to a fluid source or a suction source.

15. The device according to claim 1, wherein at least one end of at least one of the telescoping tubes includes a chamfered edge.

16. The device according to claim 1, wherein the second telescoping tube slidably fits into the first telescoping tube.

17. A minimally invasive surgical device, the device having a device proximal end and a device distal end, the device comprising:
a first telescoping tube having a first tube proximal end and a first tube distal end, the first telescoping tube being disposed on the device proximal end;
a second telescoping tube having a second tube proximal end and a second tube distal end, the second telescoping tube being disposed on the device distal end, the second tube proximal end slidably fitting into the first tube distal end;
a first absorbent member disposed on the proximal end of the first telescoping tube; and
a second absorbent member disposed on the distal end of the second telescoping tube;
wherein the first telescoping tube and the second telescoping tube can be adjusted with respect to each other to vary the overall length of the device and lock with respect to each other once the overall length has been determined; and
wherein the device includes at least one hole to allow passage of fluids through the device when the device is attached to a suction source or a fluid source.

18. The minimally invasive surgical device according to claim 17 wherein the absorbent members are of different sizes between about 5 and 12 millimeters to allow cleaning of cannulas of those sizes during minimally invasive surgery.

19. A method for cleaning a cannula in a minimally invasive surgical port comprising:
adjusting telescoping tubes having a fluid flow passage extending therethrough to a predetermined telescopic length;
inserting an end of the telescoping tubes into a surgical port cannula, the inserted end having an absorbent member associated therewith sized to rub against an inner surface of the surgical port cannula when the surgical device is inserted therein, a noninserted end of the telescoping tubes having a different sized absorbent member associated therewith; and
causing a flow of fluid through the fluid flow passage at least while the end of the telescoping tubes is inserted in the surgical port cannula.

20. The method of claim 19, wherein the flowing fluid passes through the absorbent member associated with the inserted end of the telescoping tubes while the end is inserted in the surgical port cannula.

21. The method of claim 20, further comprising activating a suctioning of fluid in the surgical port cannula through the absorbent member and the fluid flow passage while also rubbing clean the inner surface of the surgical port cannula with the same absorbent member associated with the inserted end of the telescoping tubes as the end is inserted in the surgical port cannula.

22. The method of claim 19, further comprising initiating an irrigation of the fluid flow passage with fluid from an external source while the end of the telescoping tubes is inserted in the surgical port cannula.

23. The method of claim 22, further comprising initiating an irrigation of the absorbent member associated with the inserted end of the telescoping tubes with the external source fluid in the fluid flow passage while the end is inserted in the surgical port cannula.

24. The method of claim 23, further comprising applying the external source fluid irrigated on the absorbent member to an inner surface of the surgical port cannula while also rubbing clean the inner surface of the surgical port cannula with the same absorbent member.
